(19) United States
(12) Patent Application Publication
(10) Pub. No.: US 2010/0317734 A1
(43) Pub. Date: Dec. 16, 2010

(54) FREE FATTY ACID BLENDS AND USE THEREOF

(76) Inventors: Michael Anthony Folan, Donegal Town (IE); Colm O'Brien, County Limerick (IE); Colum Dunne, County Tipperary (IE)

Correspondence Address: JACOBSON HOLMAN PLLC, 400 SEVENTH STREET N.W., SUITE 600, WASHINGTON, DC, 20004, US

(21) Appl. No.: 12/734,929

(22) PCT Filed: Dec. 4, 2008
(86) PCT No.: PCT/IE2008/000118

§ 371 Date: Aug. 30, 2010

Related U.S. Application Data
(60) Provisional application No. 60/996,754, filed on Dec. 4, 2007.

(30) Foreign Application Priority Data
Oct. 9, 2008 (IE) 2008/0819

(51) Int. Cl. A61K 31/20; A61P 31/10; A61P 31/00; A61P 17/02

(52) U.S. Cl. 514/558

(57) ABSTRACT

An antimicrobial composition comprising a blend of two or more natural free fatty acids derivable from milk serum lipid, the free fatty acids being selected from: butyric (C4); caproic (C6); caprylic (C8); capric (C10); lauric (C12); myristic (C14); palmitic (C 16): palmitoleic 5 (C16:1); stearic (C1 8); oleic (C18:1); linoleic (Cl 8:2); linolenic (C18:3); and esterified derivatives thereof, and a milk, protein as an emulsifying agent for the free fatty acids wherein at least 35% of the total lipid content comprises free fatty acids selected from one or more of: butyric, caproic, caprylic, capric and lauric.

### INTRODUCTION

This invention relates to blends of free fatty acids and uses thereof. In particular the invention relates to blends of antimicrobial free fatty acids.

Butter acids are free fatty acids (carboxylic acids) that exist in nature usually as alcohol esters, where they comprise the main components of plant and animal oils and fats. They are conventionally classified as short, medium or long chain carbon molecules with a carboxylic acid (polar) group on one terminus and a methyl (non-polar) group on the other. This polarity contributes amphipathic properties where the carboxylic acid terminus is water soluble, and the methyl terminus is fat soluble. Despite the hydrophilic acid terminus, and with the exception of very short chain acids (butyric), fatty acids are generally insoluble in water, but soluble in organic solvents and oils.

Fatty acid carbon chains are ‘saturated’ when linked with single covalent bonds where the full valency of the carbon is occupied by hydrogen in the —CH₂— conformation. Where carbons are linked by a double bond the full valency of the carbon is not occupied and the carbon is described as unsaturated; hence the tradition of describing fats as saturated or unsaturated.

It is also conventional to describe a fatty acid by the number of carbons in its molecular structure, C number, followed by the number of double bonds contained therein: C_{18:2} is linoleic acid, it has a chain length of 18 carbons with two double bonds i.e. two unsaturated C═C links. Saturated fatty acids tend to be straight chain molecules and pack tightly together, unsaturated fats are usually twisted at the point of the double bond and lie less tightly packed. As the carbon chain length increases the overall molecular weight increases and the overall melting point of the fats increases. Unsaturated fats are exceptional however, because of their (usually) non-linear conformation, they lie less tightly packed and have much lower melting points.

In plant and animal oils and fats, fatty acids are esterified to an alcohol such as glycerol (trihydric alcohol) which may be combined with 1, 2 or 3 fatty acids to give mono, di or trigycerides. In nature, these are usually mixed triglycerides with different fatty acids at each alcohol group. It follows also that the melting point of any triglyceride will be a feature of the melting points of the esterified fatty acids, and the overall hardness of the fat or oil is therefore a feature of the ratio of different fatty acids and whether these are saturated or not: triglycerides are described as oils when liquid at room temperature and fats when they are solid.

Butterfat from mammalian milk provides a broad spectrum of fatty acids, including short to medium chain acids not found in plant or vegetable sources. While there is some species variability it is generally comprised of butyric (C4), caproic (C6), caprylic (C8), capric (C10), lauric (C12), myristic (C14), palmitic (C16), stearic (C18), oleic (C18:1), linoleic (C18:2), linolenic (C18:3) and some minor concentrations of higher acids. It is notable that the only three unsaturated fatty acids are oleic, linoleic and linolenic and that these make up some 30% or less of the total; the following table (Table 1) presents a typical profile of the fatty acid content of bovine butterfat.

TABLE 1
Composition of Bovine Butterfat

| | Carbon | Melting point | |
|---|---|---|---|
| Fatty Acid | number | (° C.) | % occurrence |
| Butyric | C4:0 | −7.9 | 4 |
| Caproic | C6:0 | −3.4 | 2.1 |
| Caprylic | C8:0 | 16.7 | 1.2 |
| Capric | C10:0 | 31.4 | 2.6 |
| Lauric | C12:0 | 44 | 3.0 |
| Myristic | C14:0 | 58.5 | 10.6 |
| Palmitic | C16:0 | 63.5 | 27 |
| Palmitoleic | C16:1 | −0.5 | 2.3 |
| Stearic | C18:0 | 69.5 | 12.8 |
| Oleic | C18:1 | 4 | 26 |
| Linoleic | C18:2 | −12 | 2.3 |
| Linolenic | C18:3 | −12 | 1.6 |
| Others | C18+ | — | 6.8 |

Despite the fact that 65% of the fat content is saturated, butter is not a particular hard fat and liquefies at temperatures in the region of 37° C., part of the reason for this is that in addition to the unsaturated acids, the short to medium chain saturated acids (C:10 and below) all have melting points below 37° C. (because of their short chain length), and these make up 10% of the total fat content.

The hypercholesterolemia contribution from butterfat arises largely from the content of myristic acid and to a lesser extent from palmitic: neither stearic nor any of the shorter chain acids (C:12 and below) contribute to elevated serum cholesterol (9). Conversely, short chain acids (from C:4 to C:12) have significant potential benefits in terms of their antimicrobial properties and their readily available energy content. There is a difference in the way short chain saturated free fatty acids are absorbed and metabolised compared to longer chain acids. Short chain fatty acids released from their triglyceride core by gastric lipase are absorbed at the enterocytes and transported in the hepatic portal vein directly to the liver, where they present an immediate and potent source of energy some 2.5 times the calorific value of an equivalent weight of carbohydrate. Longer chain acids are re-assembled as triglycerides in the enterocytes and transported to adipose tissue as chylomicrons in the lymph (8).

In nature, most fatty acids exist as triglycerides, the main components of the lipid fraction of fats or oils. On ingestion lipid is broken down by lipase enzymes, releasing free fatty acids from the glyceride. The process of hydrolysis starts with the action of pre-gastric (salivary) lipase and it is known that most salivary lipases preferentially release short to medium chain acids from milk lipid, and that these short to medium chain acids have potent inhibitory properties against Gram positive enterococci and gram negative coliforms (1 & 2). In addition to the short chain acids, both linoleic and linolenic are known to have inhibitory effects against the dental caries organism, Streptococcus mutans (3). Monoglycerides of fatty acids as well as the free short chain acids are known to have anti-fungal activity (4). Viricidal activity against the enveloped viruses has also been reported (7).

Early and preferential release of microbicidal short chain fatty acids (C:4 to C:12) by salivary lipase provides a protective mechanism in the gut of the new born animal and contributes significantly to prevention of gastro-intestinal infection. Free fatty acids are generally insoluble in aqueous media and so their transport and bio-availability depends on their emulsification.

WO 03/018049 discloses the use of a milk serum apo-protein to inhibit adhesion of potential pathogens to human or animal epithelial membranes; the apo-proteins are generated enzymatically from milk serum lipo-proteins by lipases derived from porcine pancreatic extracts. Apo-proteins are the residual protein ‘back-bone’ left after the normally conjugated non-protein material (lipid/carbohydrate/polysaccharide/metal ions) has been removed. Apo-proteins normally have a great affinity for particular non-protein conjugates. In the case of apo-lipoproteins this affinity is based on polar and non-polar or amphipathic moieties that facilitate binding and transport in aqueous media of lipid, fats or free fatty acids.

During milk processing, butter manufacture involves splitting the fat globule membrane by mechanical means and allowing the butterfat globules to coalesce and float to the surface where they are collected as butter. The residual buttermilk is frequently acidified to precipitate the acid insoluble casein proteins leaving ‘acid whey’ comprising the milk serum proteins, the milk sugar lactose and constituents of the disrupted fat globule membranes. Whey proteins are also recovered from the run-off from curds in cheese making where rennet is used to coagulate the casein and fat, this ‘sweet whey’ will also contain constituents of the fat globule membranes. Whey proteins can be further processed to remove residual lactose and lipid and then spray dried to form whey protein concentrate (WPC) or whey protein isolate (WPI) when processed to minimise non-protein material

Apart from whole milk, the ‘primary’ products in commercial dairying are butter and cheese, with buttermilk and acid casein, lactose, minerals and acid whey being ‘secondary’ products from butter making while, sweet whey, minerals and lactose are ‘secondary’ products from cheese manufacture. In modern dairying, the terms primary and secondary may be misleading as secondary ‘by-products’ make a very significant economic contribution. In addition, modern dairying makes use of increasingly sophisticated technical methods to modify and increase the value of traditional products. One such modification is the use of enzymes to generate free fatty acids from butterfat to obtain cheese flavours (Enzyme Modified Cheese) for use in food processing, the manufacture of savoury snacks, and ready prepared meals.

### STATEMENTS OF INVENTION

The invention provides an antimicrobial composition comprising a blend of two or more natural free fatty acids derivable from milk serum lipid, the free fatty acids being selected from: butyric (C4); caproic (C6); caprylic (C8); capric (C10); lauric (C12); myristic (C14); palmitic (C16); palmitoleic (C16:1); stearic (C18); oleic (C18:1); linoleic (C18:2); linolenic (C18:3); and esterified derivatives thereof, and a milk protein as an emulsifying agent for the free fatty acids wherein at least 35% of the total lipid content comprises free fatty acids selected from one or more of butyric, caproic, caprylic, capric and lauric.

The total lipid content may comprise at least 50% of free fatty acids selected from one or more of: butyric, caproic, capric and lauric.

The remainder of the total lipid content of the composition may comprise non-antimicrobial free fatty acids of C14 or higher, unhydrolysed lipid components, monoglycerides. diglycerides, triglycerides and the like or combinations thereof.

The blend may comprise a mixture of caprylic acid, capric acid and lauric acid.

The melting point of the blend of free fatty acids may be less than the highest melting point of any one of the individual free fatty acids. The melting point of the blend may be less than 45° C., for example less than 37° C., such as less than 18° C.

The ratio of caprylic:capric:lauric may be about 40:30:30.

At least some of the free fatty acids in the blend may be esterified. The esterified free fatty acids may be in the mono- and/or di- and/or tri-glyceride forms. The ratio of free fatty acids: esterified glyceride may be about 50:50.

The composition may be water dispersible.

The emulsifier may be a milk serum protein, for example the emulsifier may be an apo-lipoprotein. Alternatively, the emulsifier may be casein. The emulsifier may be present in a concentration range of about 5% to 45% by weight.

The pH of the composition may be about 4.5 to 5.0.

The invention further provides for a pharmaceutical preparation comprising a composition as described herein and a carrier. The composition may be present in a concentration range of about 0.5% to 10% weight/volume.

The preparation may be in the form of a solution, a soap, a gel, a paste, an ointment, a foam, a spray, a powder, a pessary, a dressing, a tablet or a foodstuff.

The invention further provides for the use of a preparation in the prophylaxis or treatment of Candida albicans infection. The preparation may be in the form of an intravaginal cream or gel or pessary. The invention also provides for the use of a preparation in the prophylaxis or treatment of non-specific bacterial vaginosis. The preparation may be in the form of an intravaginal cream or gel or pessary. The invention further provides for the use of a preparation in the prophylaxis or treatment of skin infection. The preparation may be in a form suitable for topical application. The invention also provides for the use of a preparation in the treatment of burns. The preparation may be in a form suitable for topical application. The invention further provides for the use of a preparation in the prophylaxis or treatment of infection. The preparation may be in the form of a surgical dressing. The preparation may be in the form of a medicament for mucosal application to the eye, nose, mouth, intestine or genitalia, for example for use in the prophylaxis or treatment of a sexually transmitted disease such as one or more of gonorrhoea, syphilis, Chlamydia, herpes and HIV.

The invention also provides for the use of a preparation in the prophylaxis or treatment of gastrointestinal infection. The preparation may be in the form of a food or drink. The invention further provides for the use in the prophylaxis or treatment of oral disease. The preparation may be in the form of an oral healthcare formulation selected from a toothpaste, chewing gum, mouthwash or other dentifrice.

In a further aspect the invention provides a food supplement comprising a composition as described herein. The composition may be present in a concentration range of about 0.5% to 10% weight/volume. The invention also provides a food supplement comprising a milk protein derived emulsifier and a mixture of caprylic acid, capric acid, lauric acid, stearic acid, oleic acid, linoleic acid and linolenic acid. The food supplement may be in the form of an oil.

The food supplement may be used for reconstituting dairy milk. The dairy milk may be skimmed milk or butter milk. The reconstituted milk may comprise 20% (w/v) or less of the food supplement, such as 10% (w/v) or less of the food supplement. Reconstitution of milk using the composition described herein may enhance the levels of naturally occurring oligosaccharides, milk minerals and vitamins in the milk.

The invention further provides for reconstituted milk comprising a composition described herein. The reconstituted milk may further comprise an oligosaccharide. The reconstituted milk may further comprise milk minerals. The reconstituted milk may further comprise vitamins.

In another aspect, the invention provides a disinfectant spray comprising a non-aqueous mixture of at least two different free fatty acids selected from: caprylic acid, capric acid, lauric acid, myristic acid, palmitic acid, stearic acid and oleic acid. The spray may further comprise an organic solvent diluent.

### BRIEF DESCRIPTION OF THE DRAWINGS

The invention will be more clearly understood from the following description of an embodiment thereof, given by way of example only, with reference to the accompanying drawings, in which:

FIG. 1 is a graph illustrating the yield of free fatty acid from fresh pasturised dairy cream using different enzymes;

FIG. 2 is a graph of water partition showing the % reduction in butyric acid;

FIG. 3 is a graph illustrating the fatty acid content after fractional distillation of washed hydrolysed butterfat;

FIG. 4 is a graph illustrating the contact viability of staphylococcus aureus on exposure to various fatty acids for 2 minutes;

FIG. 5 is a graph illustrating the depression of melting point of palmitic, lauric, myristic and capric acids with an increasing percentage of caprylic acid;

FIG. 6 is a graph illustrating the depression of melting point of lauric acid with increasing concentrations of caprylic, capric, or oleic acids;

FIG. 7 is a graph illustrating the reduction of antimicrobial potency of caprylic, capric or lauric acids with an increasing concentration of oleic acid;

FIG. 8 is a graph illustrating the contact viability of Candida albicans following 2 minutes exposure to various emulsion of free fatty acids; and

FIG. 9 is a graph illustrating the effectiveness of a free fatty acid oil as a beef carcass disinfectant against enterohemorrhagic E. coli O157 (EHEC).

### DETAILED DESCRIPTION

It is possible to generate a composition of free fatty acids from butter-fat using commercially available lipase enzymes. Such enzymatic hydrolysis forms the production basis for enzyme modified cheese. The ratio and composition of the free fatty acids generated will depend on the type of enzyme, substrate concentration and other physio-chemical conditions such as temperature and pH. Fungal lipases and natural animal pre-gastric (salivary) lipase which are extracted from the sub-lingual glands of calf, kid and lamb in abattoirs are routinely used in the cheese industry for flavour generation. The particular flavour will be influenced by the choice of enzyme and the release characteristics of short to medium chain free fatty acids. Butyric acid (C4) has a particularly pungent ‘cheesy’ odour, while caproic (C6) and caprylic (C8) are also odoriferous, but more ‘heady’ while other shorter chain volatile acids (propionic) are generated indirectly through microbial action all of which contribute to the international repertoire of regional cheese flavours.

Apart from their use in flavours, short to medium chain acids (C4 to C12) have particularly potent antimicrobial effect, their medicinal use for this purpose is however limited by their insolubility in aqueous medium and more particularly their odour which is undesirable in topical application. Early release of short to medium chain free fatty acids by the hydrolytic action of salivary lipases is recognised as being part of the ‘maternally conferred’ innate immune competence that protects the neonatal gut from potential microbial pathogens (1 & 2)

We describe how the more odoriferous content of enzyme hydrolysed butterfat can be reduced by washing or ‘cutting’ the hydrolysed fat in several volumes of water. Butyric acid is relatively soluble in water and will therefore partition into an aqueous phase, as will some of the caprylic acid, although caprylic acid is only sparingly soluble in aqueous solutions. We found that the presence of butyric acid increased the solubility of caprylic acid as the butyric acid, acts as an organic solvent thereby enabling the partition of caprylic acid.

An enzyme reaction can be driven to achieve complete hydrolysis of a substrate, this is usually done by either binding the products of the enzyme reaction or removing the products from the reaction mixture. Fractional distillation of the enzyme hydrolysed fatty acid mixture can be used to remove the more volatile short chain free fatty acids, and/or the presence of an emulsifying agent such as bile acids or a whey protein isolate in the reaction mixture will serve to bind the reaction products, thereby enabling complete hydrolysis of the substrate.

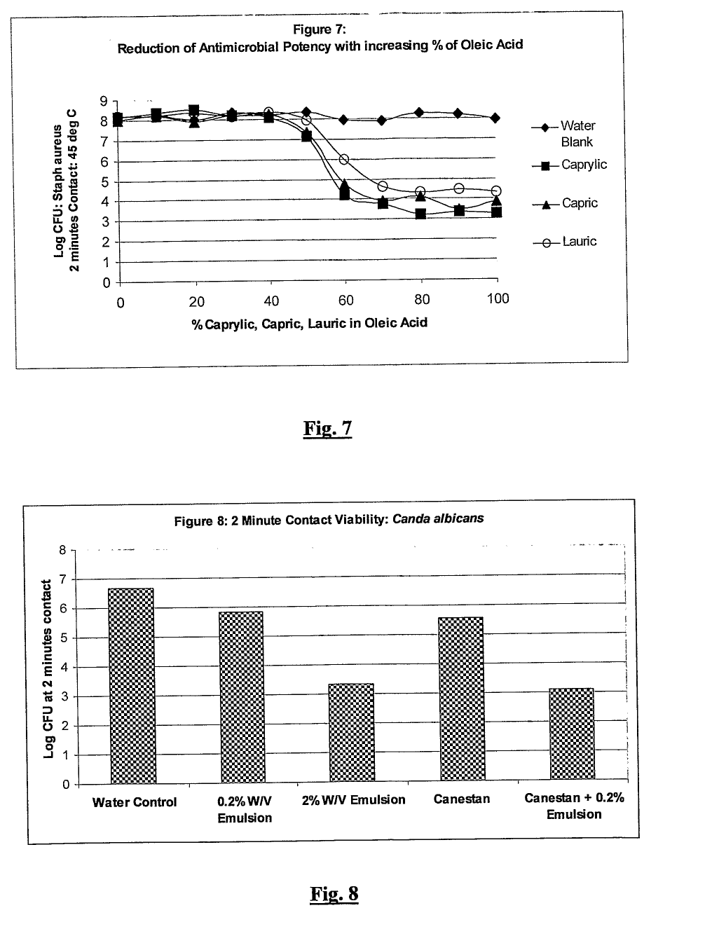

While the generation of free fatty acids by enzyme hydrolysis is relatively straightforward that the ratio of hydrolysed free fatty acid to unhydrolysed lipid is critical in achieving high antimicrobial potency. When there is an excess of unhydrolysed lipid present in the mixture, the unhydrolysed lipid acts as a sink in to which free fatty acids are sequestered due to their lipophylicity. Antimicrobial applications require a high ratio of free acid, the free acid content should be greater than about 35% of the total lipid content of the mixture. As described in more detail in Example 4, and with reference to FIGS. 6 and 7, having free fatty acids having different melting points can be combined to produce a composition having a melting point below that of the melting point of any one of the individual free fatty acid components. This depression of melting points can be used to form antimicrobial compositions of free fatty acids because individual free fatty acids exhibit an antimicrobial effect only when they are in a liquid form. Individual free fatty acids with low melting points act as solvents for acids with higher melting point, and as such free fatty acids with low melting points can act to sequester antimicrobial fatty acids and render them inactive below a certain ratio; therefore to overcome the sequestering effect, the antimicrobial free fatty acids should comprise at least 35% of the total lipid content of a composition.

Free fatty acids are insoluble in water which limits their medicinal use as antimicrobial agents. We have shown that by emulsifying compositions of free acids in aqueous based solutions, fat globules are formed having an increased surface area which results in an enhanced antimicrobial effect. Suitable emulsifying agents are milk proteins, such as milk serum proteins or milk serum apo-lipoproteins from dairy whey.

Compositions of free fatty acids are commercially available and are sold and used as ‘butter acids’ for flavouring in food production; butter acids are cleared by the United States FDA as generally recognised as safe (GRAS) for food use.

We describe formulations of butter acids that can be optimised for antimicrobial effect. Compositions of free fatty acids can be generated by enzyme hydrolysis of milk lipid, or by extracting and concentrating free fatty acids under vacuum evaporation. Blends of commercially available free acids may also be assembled when assembling blends of free fatty acids, it may be desirable to omit the more odoriferous butyric and caproic acid, particularly if the application is for topical antimicrobial purposes. For enhanced antimicrobial effect it is desirable to increase the content of the free fatty acids that are known to be antimicrobial, these being the less odoriferous caprylic, capric, and lauric acids, within a blend. We have shown that the melting point of a mixture of these three fatty acids (caprylic, capric and lauric) is a product of the combined individual melting points and that the overall melting point of the blend of the free fatty acids can be tailored by altering the ratio of the free fatty acids within the mixture.

The overall melting point of free fatty acid blends has a pivotal effect on the antimicrobial potency of these blends. A blend of caprylic, capric and lauric acid in which capric and lauric are predominant will have a higher melting point than a mix of the same acids in which caprylic is predominant. The antimicrobial effect of such blends is reduced by several orders of magnitude below the melting point of the mix; and conversely amplified by several orders of magnitude above the melting point.

The internal body temperature of a mammal is normally 37° C., whereas skin temperature can be as low as 18° C.-20° C. Where mixtures of free fatty acids are formulated to provide an antimicrobial effect on the skin, the melting point of the free fatty acid mixture should be less than about 18° C., for internal use the melting point of the mixture may be higher (i.e. the mixture should include a higher ratio of higher melting point acids), but in any event it should not exceed 37° C. if an internal (gastrointestinal) antimicrobial effect is required.

Free fatty acids have a bitter ‘peppery’ flavour and in concentrated form they are irritating to mammalian tissue. The unpleasant taste and irritant nature of free fatty acids may be ameliorated somewhat by the addition of emulsifying proteins, but free fatty acids have an acid pH which is incompatible with acid insoluble proteins such as casein and other desirable excipients such as calcium salts. In nature, fatty acids mostly exist in the form of alcohol esters, (in milk butterfat as esters of the trihydric alcohol glycerol), these are almost always mixed esters with three different fatty acids bound covalently to each glycerol. To obviate the undesirable characteristics of acidity, taste and irritability it may be desirable to convert some or all of the free fatty acids in a mixture back to mono, di or triglyceride ester form.

It is possible to re-esterify free fatty acids to glycerides using reflux distillation in glycerol and in the presence of sulphuric acid, anhydrous sodium sulphate or other suitable dessicant to facilitate the condensation reaction. Lipase enzymes, in addition to their hydrolytic activity against glycerides, act as esterases and may be used to synthesise glycerides from combinations of free fatty acids and glycerol. (5). The type of glyceride formed and the degree of esterification undergone can be controlled by varying the stoichiometric ratio of free fatty acid to glycerol; the reaction conditions; and the choice of solvent.

It is possible to construct mono, di- or triglyceride esters of individual free fatty acids and blend these in any desirable ratio, or to start with a desirable mix of free fatty acids and faun a mixed tri-glyceride ester.

While glycerides of fatty acids have much lower antimicrobial potency compared to free fatty acids, glycerides are rapidly hydrolysed to free acids on ingestion by pre-gastric salivary lipase and pancreatic lipase in the gut. Where an intestinal antimicrobial effect is desirable, for example in the prevention and/or treatment of gastroenteritis, ingestion of glycerides of short to medium chain fatty acids will result in a localised and controlled delivery of free fatty acids in the intestinal lumen without subjecting the recipient to the undesirable organoleptic properties of the free fatty acids.

We describe a process for emulsifying of selected ratios of free fatty acids, their mono, di- or triglycerides in varying combinations and ratios using commercially available whey protein isolates (milk serum proteins). Stabilisers, anti-oxidants, micro-nutrients, flavours and/or fragrances may be combined with the emulsions to provide a bulk intermediate with potent antimicrobial properties on ingestion.

Dairy milk may be reconstructed using a whey protein emulsion of tailored free fatty acids and/or their monoglycerides blend dispersed in an aqueous formulation of milk casein and milk minerals, with or without the milk sugar, lactose. It is also possible to re-construct dairy milk using tailored re-esterified free fatty acids, and/or free fatty acids in a whey protein emulsion, or as an oil by dispersing or emulsifying the free fatty acids in skim milk or buttermilk. Such formulations have substantial commercial value as they provide enhanced GI protection from food borne pathogens.

In one embodiment the invention relates to a composition of free fatty acids selected from the naturally occurring free fatty acids present in milk lipid, the composition being a combination of two or more free fatty acids selected from: butyric, caproic, caprylic, capric, lauric, myristic, palmitic, stearic, oleic, linoleic and linolenic. The milk lipid is preferably from the milk of a bovine, caprine or ovine species, but may also be from any mammalian species.

Free fatty acids may be generated by enzymatic or chemical hydrolysis of milk lipid and used as a formulation of partially hydrogenated milk lipid or they may be extracted and purified from a crude hydrolysate by fractional distillation under vacuum. Free fatty acids may also be acquired commercially and the original source of these may be from hydrolysates of plant oils such as corn, palm, palm kernel oil, coconut canola etc, but in any event they will be of natural origin and limited to those fatty acids naturally occurring in milk lipid, commercially described as butter acids.

The composition of free fatty acids may be optimised for an enhanced antimicrobial effect by increasing the ratio of butyric, caproic, caprylic, capric and lauric acid relative to the other free fatty acids, in a combination so that the overall melting point of the blend is preferably less than the normal physiological temperature of about 37° C., and in any event no more than about 45° C.

The composition of antimicrobial free fatty acids may preferably be restricted to caprylic, capric and lauric to minimise the odoriferous nature of the composition. In this regard, butyric and caproic acids may be removed from a crude hydrolysate by aqueous partition, fractional distillation, or de-selection in commercially acquired stocks.

A composition of antimicrobial free fatty acids may be used on its own or in combination with one or more of the other naturally occurring free fatty acids selected from myristic, palmitic, stearic, oleic, linoleic and or linolenic or derivatives thereof. When assembled for antimicrobial effect, the composition of antimicrobial free fatty acids (C4 to C12 free fatty acids) or derivatives thereof should preferably constitute more than about 80% of the total free fatty acid, free fatty acid derivatives and/or unhydrolysed lipid component and in any event the antimicrobial free fatty acids or derivatives thereof should not be less than about 35%, preferably 50% such as 80% of the total lipid or lipid-like material content of the composition.

In a further embodiment, selected individual free fatty acids or mixtures thereof may be esterified with glycerol, either by acid reflux condensation or enzymatically to generate mono-, di- or triglycerides which may be homogenous or mixed glycerides depending on the composition and concentration of the fatty acid feed-stock. Esterification is preferable, but not essential, where combinations of antimicrobial free fatty acids are assembled for inclusion in food products and where it is desirable to reduce the acidity, irritability and or taste of the free acids.

Re-construction of mixed triglycerides from blends of free fatty acids facilitates the re-construction of tailored lipids with enhanced utility in medical applications apart from or combined with their antimicrobial effect. A lipid based on predominantly short to medium chain fats will have a much faster metabolic energy yield based on their direct transport to the liver via the hepatic portal vein. Equally a lipid re-constructed from a selection of fatty acids that do not contain myristic or palmitic acid would be more acceptable to individuals predisposed to elevated serum cholesterol.

A composition of antimicrobial free fatty acids and/or derivatives thereof with or without excipients may be used as a spray or a wash to reduce the bio-burden of potential pathogens on animal carcass in abattoirs, and for similar reasons on freshly harvested fruit and vegetables.

It may be desirable, but not essential, to emulsify the hydrolysed lipid, free fatty acids and or derivatives thereof to increase their bio-availability for antimicrobial and or nutritional effect. Suitable emulsification agents include milk, milk proteins, milk casein, milk serum proteins, milk serum apo-proteins and milk whey from dairy processing.

Formulations of free fatty acids may be presented in the form of conventional medicines, medical devices, cosmetics or dietary components and when used as a dietary component they may be formulated to achieve a nutritional effect such as high calorific value and or an antimicrobial effect in the gut, or both.

In one embodiment the selected composition of antimicrobial free fatty acids, or glycerides thereof and/or crude hydrolysate of milk lipid is emulsified in a whey protein and used as an ingredient in ointments, lotions, gels, creams, pastes or other medicaments for topical treatment and/or prophylaxis of infections of the skin, hair, and nails.

In a further embodiment the whey protein emulsion of selected antimicrobial free fatty acids is used as an antimicrobial ingredient in toothpaste or mouthwash or denture adhesive or fixative for oral hygiene and as a medicament for treatment and/or prevention of dental caries, gingivitis, periodontitis, thrush and aphthous ulcer.

In a further embodiment the whey protein emulsion of antimicrobial free fatty acids may be incorporated in a gel or pessary and used for prevention and/or treatment of vaginal infections including yeast Vaginitis and non-specific bacterial Vaginosis and in a similar form as a barrier for prevention of sexually transmissible disease including gonorrhoea, syphilis, Chlamydia, Herpes and HIV.

Whey protein emulsions of antimicrobial free fatty acids may be used as ingredients in soaps, hand-gels and wet-wipes for prevention of cross-contamination of antibiotic resistant bacteria in hospitals and patient care establishments and may also be used for de-colonisation of known asymptomatic carriers of antibiotic resistant bacteria.

Antimicrobial compositions of free fatty acids may be emulsified as a foam using heat treated whey proteins and other excipients to prepare an enema suitable for prevention and/or treatment of infectious diarrhoea, pseudomembranous colitis and other bowel infections.

The invention will be more clearly understood from the following examples.

### Examples

Methods

Gas Chromatography Analysis of Fatty Acids

The lipid component of samples, (containing the free fatty acids) are extracted from any non-lipid material present in the original sample prior to analysis by gas chromatography (GC). The method used is adapted from DeJong and Badings (6). A sample (0.1-0.5 g) was weighed out and added to a solution containing 10 ml ethanol, 1.0 ml 2.5M sulphuric acid, 15.0 ml 50:50 di-ethyl ether:heptane and 1 ml of internal standard, (i.e. valeric (C_{5:0}), margaric (C_{7:0}) and undecanoic acid (C_{11:0}), all at 1 mg/ml in heptane). The solution was mixed well and centrifuged to recover the supernatant. The upper phase containing lipid was transferred to a beaker containing 1 g of anhydrous sodium sulphate (dessicant) using a Pasteur pipette.

Solid Phase Extraction (SPE) was carried out using 500 mg, 3 ml Strata aminopropyl columns (Phenomenex, Macclesfield, Cheshire, UK). These SPE columns were first conditioned with 2×10 ml of heptane, following which the lipid fraction from the beaker above was added to the column and drawn through by vacuum. Neutral lipids were eluted using 2×10 ml flushes of hexane:propanol (3:2 v/v); fatty acids were then eluted from the SPE column using 5 ml of 2% v/v formic acid in diethyl ether.

Individual free fatty acids (C_{4:0}, C_{6:0}, C_{8:0}, C_{10:0}, C_{12:0}, C_{14:0}, C_{16:0} C_{18:0}, C_{18:1} C_{18:2} and C_{18:3}) were quantified by GC using a Varian 3800 Gas Chromatograph with flame ionization detection, a Varian 1079 Universal capillary injector, a Varian 8410 liquid autosampler and Varian Star operating software (Varian Analytical Instruments, Harbor City, Calif.; USA). The column used was a Chrompack WCOT fused silica capillary column CP FFAP-CB, 25 m×32 mm ID, 0.3 DF (Varian Analytical Instruments, Harbor City, Calif., USA) with direct on column injection. Injector temperature was 65° C. (held for 0.1 min), increased to 250° C. at 200° C. per min (held for 1 min), followed by cooling to 65° C. at 200° C. per min (held for 20 min). Flow pressure was fixed at 8 psi. Oven temperature was 65° C. (held at 1.5 min), then heated to 240° C. at 10° C. per min, total run time 44 min. Detector temperature was 300° C. All samples were analyzed in triplicate.

Antimicrobial Analysis

Two techniques were routinely used to evaluate antimicrobial effect and to compare potency of individual samples, these were a growth inhibition assay a contact viability assay and an assay for Minimum Inhibitory Concentration (MIC). Several ‘indicator organisms were also routinely used these included a gram positive bacteria (Streptococcus mutans), a gram negative bacteria, (Salmonella typhimurium) and a yeast (Candida albicans). The test organisms were held at −80° C. in MicroBank cryoprotectant vials (Pro-Lab, Ontario, Canada) and cultured on a suitable agar prior to inoculation of liquid cultures for use in the assays: Bacteria were grown on Brain Heart Infusion agar and broth (Oxoid, UK), while yeast was grown on YEPD (from Oxoid UK, 1% W/V Yeast Extract, 1% W/V peptone, and 2% W/V glucose where required Bacteriological agar 1.8% W/V).

Contact Viability Assay

This assay procedure is used to measure the reduction in viability (survival) of a standardized microbial population exposed for defined periods of time to formulations containing free fatty acids and/or derivatives thereof with or without other active or inactive constituents that may constitute a delivery system for therapeutic or nutritional use.

The procedure is based on the enumeration of the number of Colony Forming Units (CFU's) in an inoculum, prepared from late log-phase bacteria or yeast grown in a suitable medium, and the comparison of these with the number of CFU's recovered from the same inoculum after a defined period of exposure to a defined quantity of a test substance. Enumeration of CFU's is achieved through the standard mcrobiological procedure of serial dilution and plate counting. The procedure allows a direct and comparative measure of the overall antimicrobial potency of gels, ointments and other viscous and opaque compositions in the concentration at which, they are intended to be used. The results are conventionally expressed as the reduction in log numbers of CFU's between the inoculum and the test substance

Stock cultures of the test bacteria or yeast are held on agar slopes and plates. Prior to assay broth cultures are prepared in suitable liquid media and incubated under suitable conditions to achieve late log-phase densities. Cultures are harvested by centrifugation and re-suspended in one tenth volume of their own supernatant to achieve ×10 concentrations. The ×10 inoculum is added to a 1 gram (or 1 ml) sample of test substance, mixed well and allowed to incubate for a period of time which may extend from 30 seconds to several minutes to more. At the end of the exposure period 9 ml of a suitable (non-growth supporting) diluent is added to each test sample and mixed well. Thereafter 1:10 dilutions are made in a serial fashion with aliquots of each dilution added to a sterile petri-dish. A suitable (growth supporting) agar is added at 45° C. and the aliquot of diluted cells mixed with this by swirling. Once set, the agar plates are incubated under suitable conditions (aerobic or anaerobic), and when individual colonies are visible they are counted at the dilution point showing between 30 and 300 visible colonies. The number counted, multiplied by the dilution factor is a measure of the number of viable cells in the test or control sample.

Minimum Inhibitory Concentration (MIC) Assay

The MIC of test samples against microorganisms was carried out using standard agar dilution methods. The test method is based on the National Committee for Clinical Laboratory Standard (NCCLS) documents M7-A6-Methods for Dilution Antimicrobial Susceptibility Tests.

Test samples were diluted in water from 1:2 to 1:24. and were further diluted 10-fold with melted agar medium such that the antimicrobial activity could be assessed over the concentration range 1:20 to 1:240. The agar medium used for bacteria was Mueller Hinton agar supplemented with 5% sheep's blood where necessary for good growth of the organism; fungi, including yeasts were grown on Sabouraud Dextrose Medium. Micro organisms were cultured on plates of agar medium at 35° C.±2° C. under appropriate environmental conditions for 16-24 h. After checking the viability and purity of each culture, micro organisms were suspended in 0.9% w/v saline to an optical density of McFarland standard 0.5. Suspensions of each organism were then applied to the agar plates containing the range of test substance dilutions using a multipoint inoculator which delivered approximately 0.3 μL suspension to the plate surface as a spot. This is equivalent to approximately 10⁴ colony forming units per spot. As a control to demonstrate viability of the inoculum and the ability of the agar medium to promote growth, organisms were also inoculated on medium not containing test substance. The spots of suspensions were allowed to dry in to the agar plates in a laminar air flow cabinet, the plates inverted and placed in incubators appropriate for the growth of the individual organisms ie under aerobic, micro-aerophilic or anaerobic conditions.

Plates were removed from incubators after approximately 24 to 48 h incubation until growth was clearly visible on control plates not containing test substance. In the case of these slow-growing fungi Trichophyton mentagrophytes and Trichophyton rubrum, plates were incubated for 8 days before assessment. Incubated plates were stored at 4° C. and assessed by visual inspection and using a proprietary image analysis system configured to record MIC data.

The presence or absence of growth at the inoculation spots was recorded and the MIC of LactiSAL determined against the individual micro organisms tested. The MIC of an antimicrobial substance is defined as the lowest concentration of test item that completely inhibits visible microbial growth as judged by eye disregarding a single colony or a thin haze within the area of the inoculated spot.

### Examples

### Example 1

### Lipolytic Generation of Free Fatty Acids from Butterfat

Selection of Enzymes

Six commercially available lipolytic enzyme preparations, were employed in this example: Lipomod 187 (mixed fungal source); Lipomod 338 (Penicillium roqueforti); Lipomod 621 (Candida cylindricia and Penicillium roqueforti) were obtained from from Biocatalysts Ltd, Cardiff, UK. Lipase AY 30 (from Candida rugosa) and Lipase R (Aspergillus niger) were obtained from Amano-Enzyme Europe Ltd, Chipping Norton, UK. Calf pre-gastric enzymes were obtained from Renco, New Zealand. The procedure is not confined to these particular enzymes and others such as crude porcine panvreating from Sigma UK which contains pancreatic lipase as well as protease and alpha-amylase may also be employed as well as mixtures of these to achieve an optimal yield.

Fresh pasteurised dairy cream was used as the substrate, in this example, but other sources of butterfat, vegetable or animal lipid may also be used as a source of free fatty acids through enzyme hydrolysis. Cream contains 50% w/w dry matter, 40% of which is butterfat. The amount of enzyme added to each digest was estimated in relation to the concentration of butterfat present. For example, 1% w/w dose rate of a particular lipolytic preparation was 1% of the butterfat content, e.g. 0.4 g/100 ml cream.

Hydrolysis Procedures

The hydrolysis reactions were carried out in triplicate in 250 ml Erlenmeyer flasks containing 100 ml of cream. Hydrolytic temperature in this example was set at 45° C., but the process may be conducted at any temperature giving suitable yield. The flasks and contents were brought to that temperature before the enzyme preparations were added. The flasks were incubated during the procedure in a rotary incubator (Stuart Scientific) at 150 rpm. Samples were removed (approx. 10 ml) at timed intervals and heat-treated at 85° C. for 10 min in a water bath to inactivate the enzyme; these were then subjected to Fatty Acid analysis by GC as described in the Methods.

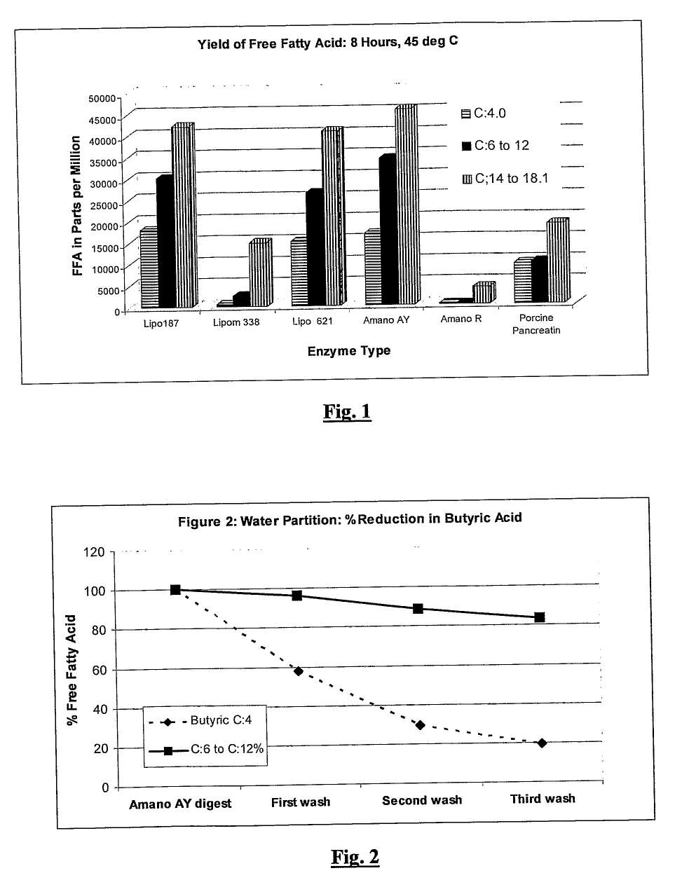

FIG. 1 shows a summary fatty acid profile after digestion with various enzymes for 8h at 45° C. (mean of three digests). The antimicrobial free fatty acids are caproic, caprylic, capric and lauric, (C:6 to C:12) and the highest yield of these were observed on digestion with Amano AY, Lipomod 187 and Lipomod 621, the lowest yield was observed when Amano R was used.

### Example 2

### Isolation and Antimicrobial Evaluation of Short to Medium Chain Free Fatty Acids from Enzyme Hydrolysed Butterfat

Water Partition

Butyric acid being the most odoriferous and least active from an antimicrobial perspective, is also the most water-soluble of the free fatty acids, and it may be separated by water partition (phase separation). Using the digest obtained from Amano AY from Example 1 above, 4 volumes of water were added at 40° C., the mixture was agitated vigorously to disperse the lipid content in the water and then left to separate (partition) at room temperature.

The water phase was decanted and two consecutive washings with 4 volumes of water were undertaken. This method removed 80% of the butyric acid and approximately 18% of caproic, caprylic, capric and lauric acids (C:6 to C:12) from the digests as illustrated in FIG. 2.

Fractional Distillation of Washed Butterfat from Third Wash Above (FIG. 2)

Methods of fractional distillation are well known to those skilled in the art, the procedure involves heating of a crude mixture of volatiles such as free fatty acids, with or without other fats, oils and or lipids, and/or other fluids such as water, organic solvents and solids. Components with the lowest boiling point will evaporate from the mixture first, followed by sequential evaporation of each higher boiling point as the temperature of the mixture is increased. Evaporated volatiles may be condensed on a cooling coil and collected in a suitable receiver. By reducing the pressure over the heated mixture in a manner most suitably achieved using a vacuum pump on a closed system of heating and cooling, the boiling point of all components is reduced by an amount that is a factor of the boiling point at atmospheric pressure, the degree of vacuum (Trouton's Equation) and the degree of association between the volatiles and other solvents known as azeotropic mixtures. In sophisticated systems the temperature at the top of the evaporating column is monitored so that collection of the condensate can commence when the required components are evaporating and condensing.

Suitable equipment for distillation of free fatty acids may be obtained from Buchi, Switzerland and include Kjeldahl, K-355 steam distillation, which is basically a method of purging a crude composition of volatiles with live steam and condensing this to obtain a mixture of volatile free fatty acids and water; the fatty acids, being insoluble, in water may be collected by decanting from the mixture, once cooled and separated.

Greater control of the distillation may be achieved using a rotary evaporator such as Buchi Rotavapor R-210 with vacuum controller V-855 The rotary evaporator allows a crude mixture in a rotating round bottom flask to be held at a constant temperature in a water bath, while a vacuum pulled on the closed system may be modified from low to medium vacuum: i.e. 3,000 to 0.1 Pascals (atmospheric pressure is 1 Bar/100,000 Pascals). As pressure decreases the boiling point of the individual components decrease and they evaporate sequentially provided an azeotropic mixture has not formed which may be prevented by the addition of other solvents.

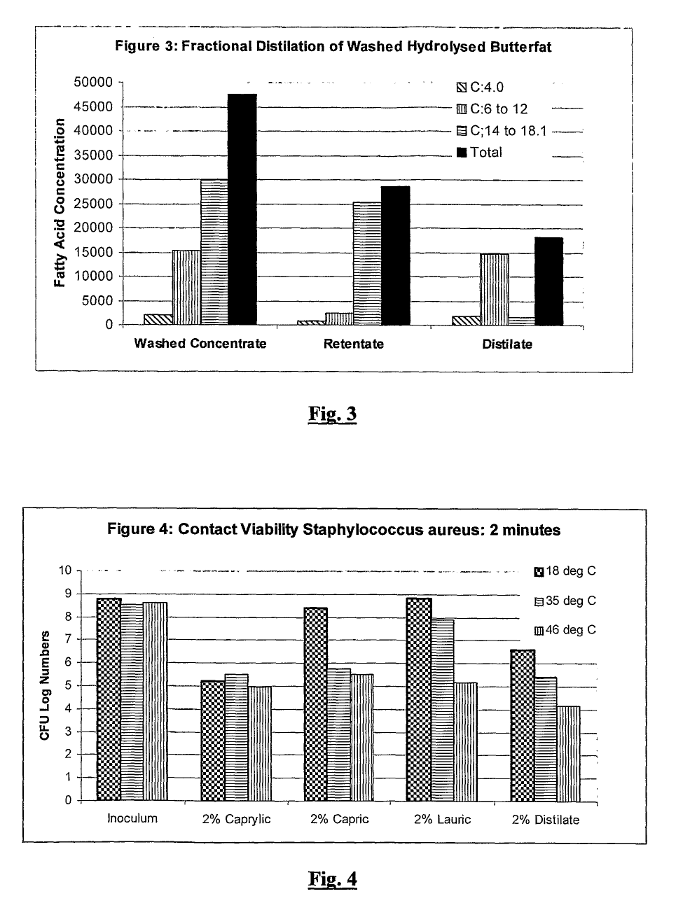

An example of fractional distillation of washed butterfat is presented in FIG. 3.

In the distillate, butyric acid was 9.8% of total, up from an original 4.5% of washed butterfat, the antimicrobial group (C:6 to C: 12) was 81% of the total distillate, up from 32% of washed concentrate, while the longer chain fatty acids (C:14 to C:18.1) were reduced from 91% to 8.5%. In terms of overall concentration, the antimicrobial group in the washed concentrate was essentially unchanged at 99%, of original, the retentate was 26% (-3.8 X) and the distillate was 251% (±2.5×).

The composition of free fatty acids (C:4 to C:12) in the distillate was:

| | |
|---|---|
| Butyric | 9.8% |
| Caproic | 5% |
| Caprylic | 10% |
| Capric | 24% |
| Lauric | 38% |

Antimicrobial Evaluation

The minimum inhibitory concentration (MIC) method described above was used to compare the relative antimicrobial potency of the concentrate, washed concentrate, retentate and distillate. In practice it proved difficult to mix the distillate with the molten agar, due to the insolubility of free fatty acids in water. The use of surfactants to facilitate solubilisation was of little value as it was discovered that these inactivate the antimicrobial effect of free fatty acids. It was therefore necessary to disperse the concentrated fatty acids in the molten agar by vigorous agitation at a temperature just above its setting point. It was anticipated that the antimicrobial potency would correlate directly to the concentration of the antimicrobial group, but surprisingly this was not the case.

TABLE 3
MIC Staphylococcus aureus (N = 3)

| | Concentration | |
|---|---|---|
| Fraction | C:6 to C:12 | MIC mg/ml |
| Concentrate | 1 | 40 |
| Washed Concentrate | 0.99 | 40 |
| Retentate | 0.26 | >100 |
| Distillate | 2.55 | 30 |

As illustrated in Table 3 the distillate was just 1.3 times more potent than the original concentrate, despite being 2.5 times more concentrated. Further evaluation suggested that the unexplained difference related to presence of milk serum proteins in the original concentrate, the partial carry over of these to the washed concentrate and their absence from the distillate. In further studies it became apparent that there was some form of synergy between the free fatty acids and other lipid or non-lipid macro-molecules of the original dairy cream, and this is illustrated in the following examples.

### Example 3

### Combination of Antimicrobial Free Fatty Acids with Lipid or Non-Lipid Components of Dairy Cream

The non-lipid components of dairy cream include milk serum proteins, lipoproteins proteo-glycans, oligosaccharides and components of the milk fat-globule membrane. It proved relatively easy to emulsify concentrations of free fatty acids with compositions of milk serum proteins (whey) and as illustrated below these gave considerably amplified antimicrobial effect. While not wishing to be bound by the explanation it would appear that milk serum components may facilitate the antimicrobial effect of free fatty acids either because some of them are amphipathic and facilitate dispersal of the insoluble fatty acids in droplets with greatly increased surface area or they have a bridging effect between the fatty acids and a microbial cell surface or both.

A suitable source of purified milk serum is a purified Whey Protein Isolate (WPI) ‘Provon 190’ that is available commercially from Glanbia PLC, Kilkenny, Ireland.

Emulsification Method 1

A 20% W/V dispersal of WPI in de-ionised water is allowed to hydrate for a period of 4 to 6 hours under constant stirring. An amount of polysorbate (Tween 20), most suitably 0.2% W/V is added, and after 10 minutes 10% V/V of a suitable salt, or solvent or other osmotic agent is added slowly under constant stirring; suitable osmotic agents may be sodium chloride, ammonium sulphate, sodium phosphate, ethanol, methanol, glycerine or lactose. The polysorbate is necessary to reduce surface tension and the osmotic agent is necessary to increase the osmolarity of the WPI solution, bringing it close to its limit of solubility. After 30 minutes a 30% V/V composition of Free Fatty Acid, such as that obtained in the distillate from Example 2 is added drop by drop into a vortex created by a high speed homogeniser. A suitable homogeniser is an Ultra-Turax Model T18 (IKA Works, Wilmington, N.C. 28405, USA) fitted with an S18N-19G dispersing tool, operating at 6,000 to 8,000 RPM. The procedure is conducted at ambient temperature (18° C. to 22° C.) and the product is a white emulsion with a consistency approximating to that of dairy cream.

Emulsification Method 2

The method described as emulsification method 1 above is most suitable for compositions of fatty acids that are liquid at ambient temperature, when it is required to prepare an emulsion of individual fatty acids or combinations that have melting points above ambient (saturated fats greater than C:8), the emulsification process must be conducted at a temperature above the individual or combined melting points: capric and lauric acids have melting points of 31.4° C. and 44° C. respectably and these are emulsified with all components at 46° C. Because a 20% W/V suspension of WPI will start to denature at temperatures above 50° C., the concentration of WPI is reduced in this method to 10% W/V.

A 10% W/V suspension of Provon 190 in de-ionised water is allowed to hydrate for 4 to 6 hours with constant stirring at ambient temperature. Unlike method 1, there is no requirement for modification of surface tension or osmolarity. The solution is equilibrated at 46° C. A 10% W/V (final volume) of lauric acid is equilibrated at 46° C., where it becomes liquid. Using a suitable homogeniser such as an Ultra Turax as described in method 1 a vortex is generated in the whey protein solution and the volume of molten lauric acid added to this vortex, rapidly in one lot. Immediately after addition of the lauric acid a volume of crushed ice equivalent to the total combined volume of whey protein and lauric Acid is added and homogenised to chill the total liquid to below 8° C. The product is a white emulsion with a consistency approximating to that of dairy milk.

Using method 1 above, the emulsified composition of free fatty acids from the distillate of Example 2, had an MIC of approximately half (potency ×2) that of the free acids on their own. The emulsion contains 30% by weight of free acid and the MIC assay dose should be adjusted accordingly to achieve an equivalent dose of free acid and emulsified acid from the test procedure.

Compared to the distillate on its own with an MIC of 30 mg/ml, the observed MIC for the emulsified distillate was 15 mg/ml, this should be adjusted by a factor of 3.3 to achieve a free acid dose equivalent of 4.5 mg/ml i.e. 6.6 times the potency of the distillate.

### Example 4

### Physical Properties of Compositions of Free Fatty Acids that Affect Their Antimicrobial Potency

Using the emulsification procedures described in Example 3 to evaluate the antimicrobial properties of varying compositions of free fatty acids, three further unexpected features were revealed:
A) Emulsified compositions of free fatty acids have little or no antimicrobial potency at temperatures below their combined melting points. An emulsion of capric acid for example has no antimicrobial effect below 31.4° C., but has a significant effect at temperature above that, such as at physiologically normal temperatures of 37° C.
B) Depending on the overall ratio, compositions of free fatty acids may have a combined melting point below that of the component with the lowest melting point; there is an apparent solvent effect which depresses the melting point of the composition.
C) Where compositions of free fatty acids include a significant proportion of non-antimicrobial free acids, or unhydrolysed lipid, there is an apparent sequestration effect. The lipophylic nature of the antimicrobial fatty acids renders them preferentially soluble in the residual lipid or non-antimicrobial free acid, which severely reduces or eliminates the antimicrobial effect.

While it is technically feasible to purify individual antimicrobial free fatty acids by fractional distillation as described in Example 2, it is more expedient to acquire these from commercial stocks as components of ‘butter acid’ blends; a suitable supplier of natural free fatty acids is Advanced Biotech Inc, New Jersey, USA.

Using emulsification method 2 described above, separate emulsions of natural caprylic, capric and lauric acid were prepared; an emulsion of the distillate from Example 2 was also prepared by method 2 and used in the following assay for comparative purposes. These ‘individual’ emulsions (and the distillate) were all prepared at 46° C. and contained 5% W/V of the individual acids compared to 30% W/V contained in emulsions prepared by method 1.

The contact viability assay method was used to evaluate the antimicrobial potency of 2% W/V suspensions of each emulsion in water at 18° C., 35° C. and 46° C. The assay was conducted in glass tubes and all test components were equilibrated at the teat temperature in a water bath before inoculation and during the suspension period (2 minutes). The inoculum is an 18 hour culture of Staphylococcus aureus grown in Brain Heart Infusion Broth. The reduction in CFU's are illustrated in FIG. 4.

FIG. 4 shows that the variation in assay temperatures from 18° C. through 35° C. to 46° C. had no significant effect on the viability of the inoculum with CFU's (N=3) being at log 8. The emulsion of caprylic acid (MP=16.7° C.) gave a 3 log reduction at all temperatures tested, while both capric and lauric had no effect at 18° C., capric gave a 3 log reduction at 35° C. and 46° C., while lauric was only effective at 46° C. The distillate has a melting point of 16° C. and gave a 2 log reduction at 18° C., a 3 log reduction at 35° C. and 4 logs at 46° C. The results illustrate that fatty acids have little if any antimicrobial effect at or below their melting point.

Combinations of Fatty Acids and Depression of Melting Points

By combining individual fatty acids of different melting points in varying ratios it is possible to build up a picture of the relative effects of the individual components on the melting point of the combination. There are a variety of commercially available apparatus for determination of melting points, but these values may also be determined by placing a volume of 50 microlitres of the molten combinations in the wells of a 96 well microtitre plate (Nunc Nalgene). The plates are chilled to below the melting point of the lowest known component (where all wells are solid), and the plate allowed to float in a bath of iced water (4° C.), and warmed up slowly with the assistance of a thermostatically controlled heater. The rate of heating should be no more than 1° C. per minute, and by careful observation the point at which wells become liquid is evident due to the change from opaque to translucent.

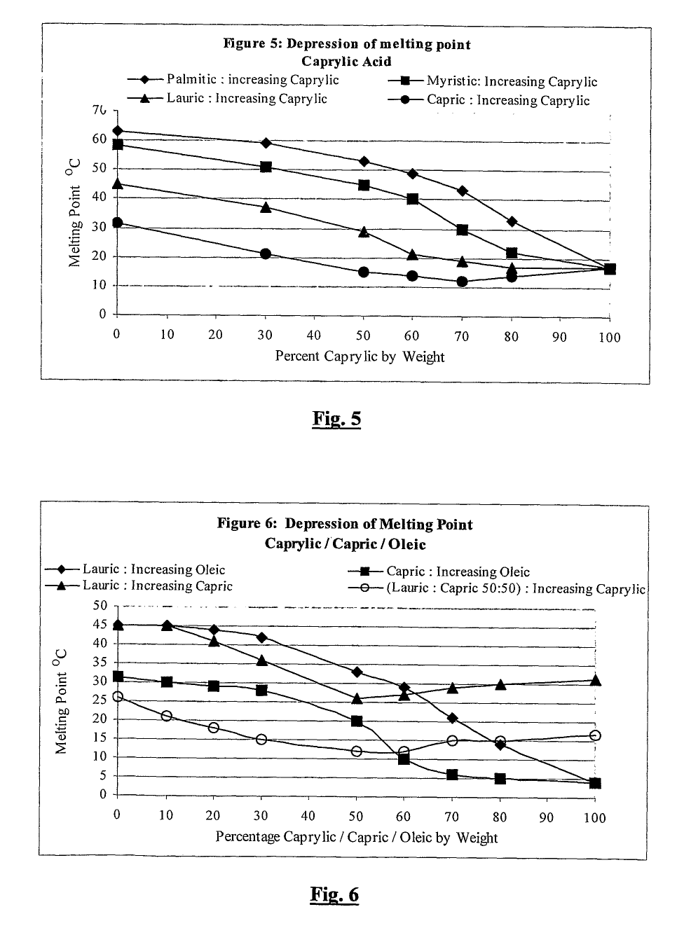

FIG. 5 illustrates the effect of an increasing ratio (W/W) of caprylic acid (MP=16.7° C.) on the melting points of palmitic (63.5° C.), myristic (58.5° C.) and lauric (44° C.)

It was anticipated that the relationship would be a straight line between the pure high melting point entity on one side and the pure low melting point entity on the other. Surprisingly, the relationship is not linear, and further there is an unexpected effect which is most easily demonstrated with two fatty acids that have relatively close melting points; in this case capric and caprylic.

As the ratio of low melting point caprylic increases from 50% through 60% and 70%, the melting point of the combination dips below the melting point of pure caprylic: the precise values are:

| | |
|---|---|
| capric 50%:caprylic 50% | 15° C. |
| capric 40%:caprylic 60% | 14° C. |
| capric 30%:caprylic 70% | 12° C. |
| capric 20%:caprylic 80% | 14° C. |
| capric 0%:caprylic 100% | 16° C. |

A similar effect is evident in the relationship between lauric and capric acid, and in the relationship between a 50:50 mix of capric: lauric with increasing concentration of caprylic, both of which are illustrated in FIG. 6. A 50:50 mix of lauric and capric acid has a melting point of 26° C., which is some 5 degrees below the melting point of capric at 31.4° C. As the ratio of capric increases to 60%, 70% and 80%, the melting point increases to 27, 29 and 30 degrees Centigrade (4, 2 and 1 degrees below the melting point of Capric acid).

As the complexity of the mix increases from two to three fatty acids a similar depression of melting point is detectable. FIG. 6 illustrates the relationship of a 50:50 mix of lauric and capric acid (melting point 26° C.) with increasing concentration of caprylic acid. The lowest melting point of 12° C. is detectable at a concentration of 50% caprylic and 50% lauric:capric (i.e. 25% lauric, 25% capric); the same value is present at 60% caprylic, and increases to 15° C. at 70% and 80% caprylic and to 16.7° C. at 100% caprylic.

FIG. 6 also illustrates the use of oleic acid as a diluent for lauric and capric acid. Oleic acid is a C 18:1 unsaturated fatty acid with a melting point of 4° C., and it was anticipated that it would have a significant effect on the melting points of lauric and capric acid. The depression of the lowest melting point was not evident with this combination, however there is a significant precipitous effect on the melting point of capric acid as the concentration of oleic acid increases above 50%.

The value of oleic acid as a diluent in combinations of antimicrobial fatty acids has limited value however as oleic acid has no significant antimicrobial effect on its own and further, it acts as a lipid ‘sink’ into which other antimicrobial fatty acids may be sequestered.

FIG. 7 illustrates the antimicrobial effect of emulsions of caprylic:oleic, capric:oleic and lauric:oleic with varying concentration in oleic acid. The emulsions were prepared by method 2 in Example 3. A 2 minute contact viability assay procedure was used to assess potency of 2% W/V in water of each emulsion against Staphylcoccus aureus; the tests were conducted at 45° C. The results indicate that potency of each of the three free fatty acids (caprylic, capric and lauric) was severely reduced where concentrations of oleic acid exceeded 60% W/V.

### Example 5

### Antimicrobial Formulations of Emulsions of Free Fatty Acids, or Glycerides Thereof, or Both

As shown in the previous Examples, it is possible to disassemble the fatty acids in milk lipid (triglycerides), and to fractionate these to selectively remove or exclude undesirable components. In particular butyric acid may be isolated from an enzyme hydrolysed fraction, or individual free fatty acids may be selected from commercial stocks of butter acids and blended together in varying ratios which facilitate manipulation of various physical and chemical properties such as hardness (melting point), odour (reduction of butyric and caproic), flavour (maintenance of low butyric) and antimicrobial effect (C:6 to C:12 ratio >50%).

It has also been shown in the previous Examples that a selected blend of individual free fatty acids may be emulsified with whey protein. These emulsions may be used to re-construct a dairy milk using the major milk protein casein and a composition of milk minerals, with an option to include lactose or replace it with other oligosaccharides such as polyols for example or longer chain polysaccharides (soluble fibre), or metabolically inert macromolecules such as polyethylene glycol or combinations of these, with or without fortification with vitamins and/or anti-oxidants, or, more simply, they may be used to re-constitute skim milk.

By their nature free fatty acids have an acid pH and the major milk protein, casein is characterised as an acid insoluble protein that will readily precipitate below pH 4.5. If a composition of free fatty acids is added directly to a solution of casein it rapidly clots (curdles) and it would be reasonable therefore to assume that these two were incompatible. In fact when free fatty acids are emulsified in whey protein, they are effectively compartmentalised and their acid pH effect minimised so that an amount of up to 20% W/V of a concentrated emulsion prepared by method 1 in Example 3 may be added to skim milk without any deleterious effect on the casein content.

Table 4 provides a list of the fatty acids and their ratios in normal dairy milk and one example of a ratio of selected natural free fatty acids from commercial stocks that have amplified antimicrobial effect and have physical characteristics (hardness) approximating to the normal composition by virtue of their increased content of lauric and stearic acid and slightly reduced oleic acid.

Compositions of fatty acids such as those listed in Table 4 will impart a sharp or bitter taste to re-constituted milk and if required this can be ameliorated by converting these free acids to mono, di or tri-glycerides using enzymatic re-esterification procedures that are adequately described by Yang et al., (5). It should be noted that acyl esterase enzymes also act as lipases and the extent of re-esterification depends on the stoicheiometric ratio of fatty acids and glycerol and also that the type of mixed glyceride will be a feature of the molar ratio in any mixture of fatty acids, not on the weight percent ratio.

TABLE 3
Re-esterified Antimicrobial Milk Fat

| | | Weight % | Weight % Improved |
|---|---|---|---|
| Fatty | Carbon | Normal | Antimicrobial |
| Acid | number | occurrence | Composition |
| Butyric | C4:0 | 4 | — |
| Caproic | C6:0 | 2.1 | — |
| Caprylic | C8:0 | 1.2 | 7.0 |
| Capric | C10:0 | 2.6 | 10.5 |
| Lauric | C12:0 | 3.0 | 17.5 |
| Myristic | C14:0 | 10.6 | |
| Palmitic | C16:0 | 27 | |
| Stearic | C18.0 | 12.8 | 35.1 |
| Oleic | C18:1 | 26 | 21.1 |
| Linoleic | C18:2 | 2.3 | 5.3 |
| Linolenic | C18:3 | 1.6 | 3.5 |
| Others | C18+ | 6.8 | 0 |

Re-Constituted Antimicrobial Skim Milks

When prepared as an emulsion in whey protein by method I in Example 3, using sodium chloride to adjust the osmolarity, the revised composition of free fatty acids in column 4 of Table 4 may be added to skim milk to replace the original milk-fat, achieving a milk product with enhanced antimicrobial effect.

A commercial powdered skim-milk, under the brand name, Marvel, is available from Premier International Foods (UK) Ltd, Spalding, Lincolnshire, England, which may be used to illustrate the compatibility of the emulsified free fatty acids with other milk components including the acid insoluble casein.

TABLE 4
Re-constituted Antimicrobial Milk

| | | | Enhanced |
|---|---|---|---|
| | % W/V | Re-hydrated | Antimicrobial |
| Component | Whole milk | Marvel | milk from Marvel |
| Protein (Casein) | 4.0-4.5 | 3.6 | 3.6 |
| Protein (whey) | (included above) | — | 1.2 |
| Carbohydrate | 5.0-5.8 | 5.3 | 5.3 |
| (lactose) | | | |
| Fat (lipid) | 3.4-4.4 | 0.05 | 0.05 |
| Emulsion of FFA | — | — | 10* = 3.3% FFA |
| Minerals | 0.2-0.4 | 0.01 | 0.01 |
| pH | 6.5-6.8 | 6.5 | 5.5 |

*10% W/V Emulsion prepared by Method 1 provides 3.3% FFA and 1.2% whey protein

### Example 6

### Antimicrobial Emulsions of Free Fatty Acid or Glycerides or Both

Based on considerations detailed in Example 4, compositions of free fatty acids may be constructed from commercial stocks and the ratio of these optimised for antimicrobial effect. An example of a suitable antimicrobial composition is presented in Table 5, it has a melting point of 10° C. and a ratio of antimicrobial fatty acids to non-antimicrobial acids of 3:1

TABLE 5
Antimicrobial Composition of Free Fatty Acids

| Free Fatty Acid | Weight % |
|---|---|
| Caprylic | 33 |
| Capric | 21 |
| Lauric | 21 |
| Myristic | 2.9 |
| Palmitic | 6.7 |
| Stearic | 1.0 |
| Oleic | 14.3 |

When emulsified with whey protein by method 1 Example 3, the emulsion has a consistency approximating to that of fresh dairy cream and a pH of 4.2 to 4.8. Using the MIC technique described in the methods, the emulsion can be shown to exhibit potent antimicrobial effect against a wide range of microbial species including gram negative and positive bacteria, yeast and fungi: the data is presented in Table 6. Most microbial species are sensitive to dilutions of 1:240 (0.4% W/V) including the antibiotic resistant Staphylococcus aureus MRSA, the most resistant species are Pseudomonas at 1:60 (1.6% W/V) and E. coli at 1:100 (1% W/V). Antimicrobial formulations containing the emulsion at between 0.5% W/V and 10% W/V inclusion will therefore offer a desirable antimicrobial effect, which may be used in both a therapeutic and prophylactic manner against pathogens and opportunistic colonisers of human and animal tissue that may be pathogenic when an individual's normal immune competence is debilitated for any reason, or that may be non-pathogenic but undesirable for cosmetic reasons.

TABLE 6
MIC Data for an Emulsion of Free Fatty Acids

| | Strain | LactiSAL MIC | Gram +ve | Gram −ve | Yeast/ |
|---|---|---|---|---|---|
| Microbial Species | Code No. | (dilution) | Bacterium | Bacterium | Fungi |
| Brevibacterium epidermidis | NCTC 11083 | >1:240 | * | | |
| Brevibacterium epidermidis | NCTC 11084 | >1:240 | * | | |
| Corynebacterium xerosis | NCTC 11861 | >1:240 | * | | |
| Corynebacterium jeikeium | NCTC 11915 | >1:240 | * | | |
| Candida albicans | ERI 5696 | >1:240 | | | * |
| Candida guilliermondii | Cg 112 | >1:240 | | | * |
| Campylobacter upsaliensis | NCTC 12206 | >1:240 | | * | |
| Campylobacter lari | NCTC 11458 | >1:240 | | * | |
| Salmonella typhimurium | NCTC 74 | 1:120 | | * | |
| Salmonella enteridis | ATCC 4931 | 1:100 | | * | |
| Escherichia coli | ATCC 884 | 1:120 | | * | |
| Escherichia coli | ATCC 11698 | 1:100 | | * | |
| Pseudomonas aeruginosa | ATCC 27853 | 1:60 | | * | |
| Pseudomonas aeruginosa | NCTC 10781 | 1:100 | | * | |
| Propionibacterium acnes | NCTC 737 | >1:240 | * | | |
| Bacteroides fragilis | ATCC 43858 | >1:240 | | * | |
| Bacteroides fragilis | ATCC 43859 | >1:240 | | * | |
| Bacteroides fragilis | NCTC 9344 | >1:240 | | * | |
| Clostridium difficile | ATCC 43598 | >1:240 | * | | |
| Clostridium perfringens | ATCC 43150 | >1:240 | * | | |
| Fusobacterium alocis | ATCC 35896 | >1:240 | * | | |
| Fusobacterium nucleatum | NCTC 10562 | >1:240 | * | | |
| Enterococcus faecium | EFAM 2 | >1:240 | * | | |
| Enterococcus faecalis | EFAS 1 | >1:240 | | * | |
| Staphylococcus aureus | MSSA 1 | >1:240 | | * | |
| Staphylococcus aureus | MRSA 3 | >1:240 | | * | |
| Streptococcus mutans | NCTC 10449 | >1:240 | | * | |
| Micrococcus lylae | NCTC 11037 | >1:240 | | * | |
| Micrococcus luteus | NCTC 7503 | >1:240 | | * | |
| Trichophyton rubrum | NCPF 295 | >1:240 | | | * |
| Trichophyton mentagrophytes | NCPF 425 | >1:240 | | | * |

ATCC = American Type Culture Collection (USA);
NCTC = National Collection of Type Culture (UK);
NCPF = National Collection of Pathogenic Fungi (UK);
Other strain no. designations refer to clinical isolates from hospital laboratories.

Antimicrobial Formulations Based on Emulsified Free Fatty Acids

The antimicrobial emulsion described in this example may be used in concentrated form or in dilutions at between 0.5% W/V and 10% W/V in aqueous formulations with or without excipients, humectants, emollients, acidity regulators, anti-oxidants, preservatives, fragrances and/or other antimicrobial substances including disinfectants, antiseptics, antibiotics, anti-fungals anti-protozoans and anti-virals with which it has a potentiating effect.

The anti-microbial emulsion should be dispersed in aqueous media with the aid of a homogeniser of the type described in Example 3. Once dispersed the emulsion will have a tendency to settle over time and this can be prevented with the use of a gel to increase the viscosity of the medium. There are many conventional polymers that are commonly used in pharmaceutical or cosmetic formulations and these are well known to those skilled in the art; derivatised cellulose polymers are commonly used, and these include, but are not limited to methyl, ethyl and propyl derivatives such as carboxymethyl cellulose, hydroypropyl and hydroxyethyl cellulose, that are available from The Dow Chemical Company, USA. Some conventional excipients are not compatible with emulsions of free fatty acids and these include most surfactants that are antagonistic to fats, lipids and free fatty acids. The optimal pH for antimicrobial effect of fatty acids is in the region of 4.5 to 5.0 and this can be maintained transiently in the use of a formulation, by the inclusion of a buffer such as 0.01 M to 0.1 M sodium citrate, even where the normal physiological pH is in the region of 7.0.

The potency and efficacy of many conventional antimicrobials, including antibiotics, has been eroded over time in use, primarily due to the emergence of resistance (or partial resistance) to these compounds by the target microbial species. There are many examples of this including the emergence of Methicillin Resistant Staphylococcus aureus (MRSA), Vancomycin Resistant Enterococcus faecalis (VREF) and many other nosocomial infections that are a major cause of concern in modern healthcare practice.

An example of an antimicrobial that has a partial inhibitory effect on its target species is the azole compound Clotrimazole, commonly used to treat infections of the yeast Candia albicans. One over the counter (OTC) formulation of Clotrimazole is Canestan from Bayer AG, Germany, incorporating the anti-fungal at 1% to 2% W/V. Using the contact viability method described in the Methods Section to evaluate the microbicidal effect of 1% Canestan cream against a fresh clinical isolate of Candida albicans it is shown in FIG. 8, that the formulation will reduce viability by 1 log order over 2 minutes: a 1% aqueous dispersal of free fatty acid emulsion on its own will reduce viability by 3 logs, while the addition of 0.2% W/V free fatty acid emulsion to Canestan affects an increase in anti-fungal potency from 1 to 3 logs; 0.2% W/V of the emulsion on its own reduces viability by just under I log.

A simple gel formulation of a free fatty acid emulsion may be constructed using carboxymethyl cellulose polymer at 3% W/V, which is first triturated with 5% W/V glycerine BP. 84 mls (volume %), of 0.1 Molar sodium citrate at pH 4.5 is heated to 45° C. into which 5% W/V Polyethylene glycol 900 is dissolved, 1% W/V phenoxyethanol is added to this and dispersed by homogenisation, following which 2% W/V free fatty acid emulsion is added and also dispersed by homogenisation. The suspension of polyethylene glycol, phenoxyethanol and free fatty acid emulsion is then added to the triturated carboxymethyl cellulose in glycerine and stirred well to disperse; the gel will require some 60 minutes to fully hydrate.

Using similar methods and provided due consideration is given to compatibility of excipients a wide variety of medicaments may be constructed incorporating free fatty acid emulsions including but not limited to gels, creams, pastes, ointments, lotions, foams, sprays, suppositories, pessaries and wound dressings, which have application in dermal, mucosal, optical, aural, nasal, oral, dental, gastro-intestinal and vaginal healthcare.

### Example 7

### Anti-Microbial Applications of Non-Emulsified Compositions of Free Fatty Acid

While there are several desirable reasons for using an emulsion of free fatty acid in nutritional and healthcare applications, the non-emulsified oil in concentrated form, or diluted in organic solvents including organic acids, has specific application in surface disinfection and particularly in reduction of bio-burden in food processing; with some exceptions such as acute intervention in shingles, free fatty acids in concentrated form are generally too aggressive for routine healthcare use.

In food processing and particularly in abattoirs where food animals are slaughtered there is great potential for contamination of the fresh carcass by intestinal contents. Such faecal matter is frequently contaminated by Salmonella species and Enterohemorhagic E. coli O157 (EHEC). Such food borne pathogens are a source of great hazard in both commercial food processing and in domestic kitchens; EHEC is potentially fatal in children under 5 years. Antimicrobial compositions of free fatty acids are particularly suitable for use in carcass treatment as they are essentially food grade ‘butter acids’ and may be applied as a spray to the entire surface of freshly slaughtered and dressed beef, pig, lamb and poultry, game and fish.

The free fatty acid composition presented in Table 5 is an oil and may be sprayed on fresh meat or food processing surfaces to achieve potent disinfection. The same oil may be usefully modified to enhance its antimicrobial effect by reducing the overall ratio of oleic acid and lauric acid pro-rata (to maintain a low melting point). Equally the oil may be diluted by up to 50% with concentrated lactic acid or other organic acid without significant loss of antimicrobial potency.

In beef carcass wash tests an oil of free fatty acids was sprayed onto sections of fresh beef, which had been infected with late log phase cultures of E. coli O157:117 (EHEC). The bacteria were allowed to adhere to the beef sections for 30 minutes after which the spray was applied to all test sections; controls were sprayed with distilled water only. Treated beef sections were macerated and evaluated for residual bacteria after 60 minutes.

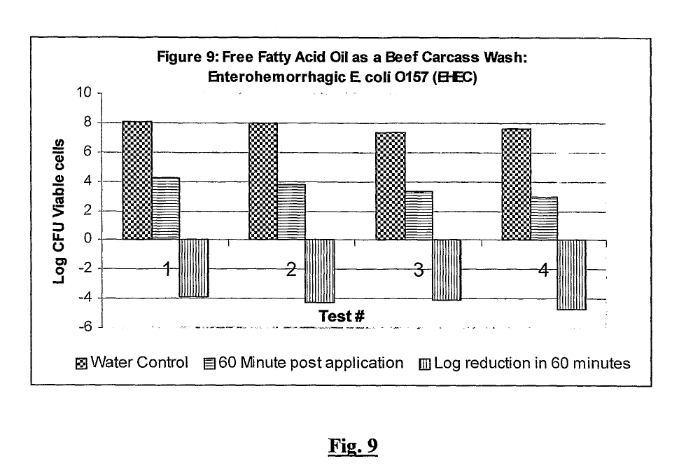

In FIG. 9, four separate tests using E. coli O157:H7 are illustrated. Residual E. coli in water washed controls was up to 8 logs of viable cells per square inch, after one hour. The treated sections show 4 to 5 (99.99%) log reductions in viable cells recovered through maceration followed by serial dilution and plate counting. Similar results have been obtained for other bacteria including Salmonella species.

The invention is not limited to the embodiment hereinbefore described, which may be varied in construction and detail.

### REFERENCES

1. Cynthia Q Sun et al (Chemico-Biological Interactions 140 (2002), pp 185-198).
2. R. Corinne Sprong et al (Antimicrobial Agents and Chemotherapy, April 2001, pp 1298-1301).
3. Schuster et al (Pharmacology and Therapeutics in Dentistry 5: pp 25-33; 1980)
4. Gudmundur Bergsson et al (Antimicrobial Agents and Chemotherapy, November 2001, pp 3209-3212).
5. Yong-Ching Yang et al. J. Chin. Inst. Chem. Engrs., Vol 34, No 6, 617-623. A Process for Synthesis of High Purity Monglyceride
6. Catrienus DeJong and Herman T. Badings 1990. Determination of free fatty acids in milk and cheese, procedures for extraction, clean up and capillary gas chromatographic analysis. Journal of high resolution chromatography, Vol. 13, February 1990. Pages 94-98.
7. Halldor Thormar et al (Antimicrobial Agents and Chemotherapy; January 1987, pp 27-31) This author reports the growth inhibitory properties of various free fatty acids against Enterococci, which are gram-positive, and coliform bacteria, which are gram-negative
8. Christopher Beerman et al (Lipids in Health and Disease 2003, 2:10) Short term Effects of dietary medium-chain fatty acids and N-3 long-chain polyunsaturated fatty acids on the fat metabolism of Healthy volunteers.
9. P. L. Zook, J. H. de Vries and M. B. Katan. Impact of Myristic acid Vs Palmitic acid on serum lipid and lipoprotein levels in healthy women and men. J Am Heart Association: Arterioscloerosis, Thrombosis and Vacular Biology: 1994;14;567-575

1-52. (canceled)

53. An antimicrobial composition comprising a blend of two or more natural free fatty acids derivable from milk serum lipid, the free fatty acids being selected from: butyric (C4); caproic (C6); caprylic (C8); capric (C10); lauric (C12); myristic (C14); palmitic (C16); palmitoleic (C16:1); stearic (C18); oleic (C18:1); linoleic (C18:2); linolenic (C18:3); and esterified derivatives thereof, and a milk protein as an emulsifying agent for the free fatty acids wherein at least 35% of the total lipid content comprises free fatty acids selected from one or more of: butyric, caproic, caprylic, capric and lauric.

54. The composition as claimed in claim 53 wherein at least 50% of the total lipid content comprises free fatty acids selected from one or more of: butyric, caproic, caprylic, capric and lauric.

55. The composition as claimed in claim 53 wherein the blend comprises a mixture of caprylic acid, capric acid and lauric acid.

56. The composition as claimed in claim 53 wherein the melting point of the blend of free fatty acids is less than the highest melting point of any one of the individual free fatty acids.

57. The composition as claimed in claim 53 wherein the melting point of the blend is less than 45° C.

58. The composition as claimed in claim 53 wherein the melting point of the blend is less than 37° C.

59. The composition as claimed in claim 53 wherein the melting point of the blend is less than 18° C.

60. The composition as claimed in claim 55 wherein the ratio of Caprylic:Capric:Lauric is about 40:30:30.

61. The composition as claimed in claim 53 wherein at least some of the free fatty acids are esterified.

62. The composition as claimed in claim 61 wherein the esterified free fatty acids are in the mono- and/or di- and/or tri-glyceride forms.

63. The composition as claimed in claim 61 wherein the ratio of free fatty acids:esterified glyceride is about 50:50.

64. The composition as claimed in claim 53 wherein the composition is water dispersible.

65. The composition as claimed in claim 53 wherein the emulsifier is a milk serum protein.

66. The composition as claimed in claim 65 wherein the emulsifier is an apo-lipoprotein.

67. The composition as claimed in claim 53 wherein the emulsifier is casein.

68. The composition as claimed in claim 53 wherein the emulsifier is present in a concentration range of about 5% to 45% by weight.

69. The composition as claimed in claim 53 wherein the pH of the composition is about 4.5 to 5.0.

70. The pharmaceutical preparation comprising a composition as claimed in claim 53 and a carrier.

71. The pharmaceutical preparation as claimed in claim 70 wherein the composition is present in a concentration range of about 0.5% to 10% (w/v).

72. The preparation as claimed in claim 70 in the form of a solution, a soap, a gel, a paste, an ointment, a foam, a spray, a powder, a pessary, a dressing, a tablet or a foodstuff.

73. The use of a preparation as claimed in claim 70 for the prophylaxis or treatment of Candida albicans infection.

74. The use as claimed in claim 73 wherein the preparation is in the form of an intravaginal cream or gel or pessary.

75. The use of a preparation as claimed in claim 70 for the prophylaxis or treatment of skin infection.

76. The use of a preparation as claimed in claim 70 for the treatment of burns.

77. The use of a preparation as claimed in claim 70 for the prophylaxis or treatment of infection.

78. The use of a preparation as claimed in claim 70 in the form of a medicament for mucosal application to the eye, nose, mouth, intestine or genitalia.

79. The use as claimed in claim 70 for the prophylaxis or treatment of gastrointestinal infection.

80. The use as claimed in claim 70 for the prophylaxis or treatment of oral disease.

81. The food supplement comprising a composition as claimed in claim 53.

82. The food supplement as claimed in claim 81 wherein the food supplement is in the form of an oil.

83. The use of a food supplement as claimed in claim 81 for reconstituting dairy milk.

84. The reconstituted milk comprising a composition as claimed in claim 53.

85. A disinfectant spray comprising a non-aqueous mixture of at least two different free fatty acids selected from: caprylic acid, capric acid, lauric acid, myristic acid, palmitic acid, stearic acid and oleic acid.
